(19) United States
(12) Patent Application Publication
(10) Pub. No.: US 2008/0281431 A1
(43) Pub. Date: Nov. 13, 2008

(54) RESORBABLE BONE GRAFT MATERIALS

(75) Inventor: Nicholas Missos, Winona Lake, IN (US)

Correspondence Address: HARNESS, DICKEY & PIERCE, P.L.C., P.O. BOX 828, BLOOMFIELD HILLS, MI, 48303, US

(73) Assignee: Biomet Manufacturing Corp., Warsaw, IN (US)

(21) Appl. No.: 11/746,978

(22) Filed: May 10, 2007

(51) Int. Cl. A61F 2/28

(52) U.S. Cl. 623/23.56; 623/23.6

(57) ABSTRACT

Ceramic materials operable to repair a defect in bone of a human or animal subject comprising a porous ceramic scaffold having a bioresorbable coating, and a carrier comprising denatured demineralized bone. The ceramic may contain a material selected from the group consisting of hydroxyapatite, tricalcium phosphate, calcium phosphates, calcium carbonates, calcium sulfates, and combinations thereof. The compositions may also contain a bone material selected from the group consisting of: bone powder, bone chips, bone shavings, and combinations thereof. The bioresorbable coating may be, for example, demineralized bone matrix, gelatin, collagen, hyaluronic acid, chitosan, polyglycolic acid, polylactic acid, polypropylenefumarate, polyethylene glycol, or mixtures thereof.

### INTRODUCTION

The present technology relates to porous ceramic structures containing a bone material, for use in repairing bone defects.

A variety of bone repair or reconstruction techniques are known in the art for repairing bone defects caused by trauma, pathological disease, surgical intervention, birth defects, or other situations where bone is inadequate for cosmetic or physiologic purposes. Such techniques can be performed using various pastes, gels, or putty-like materials, such as those containing collagen, natural bone materials and ceramics.

Many such compositions are prepared from demineralized bone matrix (DBM). Demineralized bone matrix is dry and can be difficult to manipulate, handle, and shape. To facilitate placement, the demineralized bone matrix may be hydrated and made malleable. The malleable demineralized bone matrix is molded by the surgeon to fit into the proper configuration of the bone defect site. If the composition is not immediately placed in the defect site, however, it may begin to dry and become brittle. Furthermore, depending on the formulation, some demineralized bone matrix compositions may not retain their shape upon drying (i.e. crumble) and thereby may lose structural integrity prior to application at the defect site. It would be advantageous to provide a material for bone repair that promotes rapid and complete bone repair, yet is easy to use during surgical techniques.

### SUMMARY

The present technology provides ceramic materials operable to repair a defect in bone of a human or animal subject. Such materials comprise a porous ceramic scaffold having a bioresorbable coating, and a carrier comprising denatured demineralized bone. The ceramic may contain a material selected from the group consisting of hydroxyapatite, tricalcium phosphate, calcium phosphates, calcium carbonates, calcium sulfates, and combinations thereof. The compositions may also contain a bone material selected from the group consisting of: bone powder, bone chips, bone shavings, and combinations thereof. The bioresorbable coating may be, for example, demineralized bone matrix, gelatin, collagen, hyaluronic acid, chitosan, polyglycolic acid, polylactic acid, polypropylenefumarate, polyethylene glycol, or mixtures thereof.

In one embodiment, the present technology provides formed compositions comprising (a) a ceramic scaffold, having porosity of from about 150 microns to about 800 microns, comprising hydroxyapatite, tricalcium phosphate, calcium phosphates, calcium carbonates, calcium sulfates, or combinations thereof, (b) a bone material comprising bone powder, bone chips, bone shavings, or combinations thereof, and (c) a carrier comprising denatured demineralized bone; wherein a surface of the ceramic scaffold is coated with a bioresorable material comprising demineralized bone matrix, gelatin, collagen, or mixtures thereof; and the composition is formed into a shape suitable for administration to the bone.

The present technology also provides methods for making a bone repair composition. Such methods comprise: (a) mixing a demineralized bone material and water; (b) heating the mixture of demineralized bone material and water to form a carrier; (c) coating a bioresorbable material onto a surface of a porous ceramic scaffold to form a coated ceramic scaffold; (d) forming a moldable composition comprising the carrier and the coated ceramic scaffold; and (e) removing moisture from the moldable composition to provide the dried bone repair composition.

Further areas of applicability will become apparent from the description provided herein. It should be understood that the description and specific examples are intended for purposes of illustration only and are not intended to limit the scope of the present disclosure.

### DRAWINGS

The present technology will become more fully understood from the detailed description and the accompanying drawings, wherein:

FIGS. 1A-1F depict shapes of porous ceramic scaffolds according to various embodiments of the present technology;

FIGS. 2A-2C depict troughs of porous ceramic scaffolds according to various embodiments of the present technology;

FIG. 3 depicts a filled trough according to various embodiments of the present technology; and

FIGS. 4A-4B depict the repair of a knee defect with a porous ceramic scaffold according to one embodiment of the present technology.

It should be noted that the figures set forth herein are intended to exemplify the general characteristics of materials and methods among those of this technology. These figures may not precisely reflect the characteristics of any given embodiment, and are not necessarily intended to define or limit specific embodiments within the scope of this technology.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. The following definitions and non-limiting guidelines must be considered in reviewing the description of the technology set forth herein.

The headings (such as “Introduction” and “Summary”) and sub-headings (such as “Carriers”) used herein are intended only for general organization of topics within the disclosure of this technology, and are not intended to limit the disclosure of the technology or any aspect thereof. In particular, subject matter disclosed in the “Introduction” may include novel technology, and may not constitute a recitation of prior art. Subject matter disclosed in the “Summary” is not an exhaustive or complete disclosure of the entire scope of the technology or any embodiments thereof.

The citation of references herein and during prosecution of patent applications regarding this technology does not constitute an admission that those references are prior art or have any relevance to the patentability of the technology disclosed herein. All references cited in the Description section of this specification are hereby incorporated by reference in their entirety.

The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific examples are provided for illustrative purposes of how to make, use and practice the compositions and methods of this technology and, unless explicitly stated otherwise, are not intended to be a representation that given embodiments of this technology have, or have not, been made or tested.

As used herein, the words “preferred” and “preferably” refer to embodiments that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that a recitation that an embodiment can or may comprise certain elements or features does not exclude other embodiments of the present technology that do not contain these elements or features.

The present technology involves the treatment of bone defects in humans or other animal subjects. Specific materials to be used in the technology must, accordingly, be biomedically acceptable and biocompatible. As used herein, such a “biomedically acceptable” component is one that is suitable for use with humans and/or animals without undue adverse side effects (such as toxicity, irritation, and allergic response) commensurate with a reasonable benefit/risk ratio. As used herein, such a “biocompatible” component is one that is suitable for use with humans and/or animals without undue adverse side effects (such as toxicity, irritation, and allergic response) commensurate with a reasonable benefit/risk ratio.

### Coated Ceramic Scaffold

The present technology provides bone repair compositions comprising a ceramic scaffold. The scaffold may be made of any biocompatible ceramic material, such as hydroxyapatite, calcium phosphates such as tricalcium phosphate, calcium sulfates, calcium carbonates, and mixtures thereof. In one embodiment, the ceramic scaffold comprises coralline hydroxyapatite, such as disclosed in U.S. Pat. No. 4,976,736, White et al., issued Dec. 11, 1990. The ceramic scaffold may also comprise tricalcium phosphate, or mixtures of tricalcium phosphate and hydroxyapatite.

The ceramic scaffold is preferably porous, having an internal void volume. The pores are defined by pore walls or internal surfaces of the ceramic scaffold. Pores may be of a selected depth and width and can be of a uniform size, a collection of different sizes, or randomly sized. The pores can partially transverse the ceramic scaffold or can be distributed through an entire region of the ceramic scaffold including a surface thereof. The pores can be interconnected so as to form a continuous flow path or channels throughout the ceramic scaffold. In one embodiment, the ceramic scaffold is sponge-like, comprising a plurality of different sized pores which are continuous and interconnected.

The ceramic scaffold can be high porosity having from about 70% to about 95% pore volume or lower porosity having from about 25% to about 70% pore volume. In various embodiments, the porosity is from about 40% to about 75% pore volume, from about 45% to about 55% pore volume, or from about 60% to about 70% pore volume.

It is understood that the size of pores can be selected based on such factors as the bone repair composition, desired end weight, desired porosity, and intended usage. The pores may range from about 1 to about 1000 microns in diameter or from about 300 to about 800 microns, preferably having a median pore size of from about 100 to about 800 microns. In one embodiment, the pore size is from about 180 to about 220 microns. In another embodiment, the pore size is from about 280 to about 770 microns, preferably having a median pore size of about 500 microns. Exemplary ceramic materials include those sold under the trade name ProOsteon, such as ProOsteon®200 and ProOsteon®500, by Biomet Osteobiologics, Inc. (Parsippany, N.J.), and Calcigen® PSI, sold by Biomet, Inc (Warsaw, Ind.).

The compositions of the present technology further comprise a bioresorbable material coated on a surface of the ceramic scaffold. Bioresorbable materials include demineralized bone; hyaluronic acid; tissue-derived proteins and other polymers, such as gelatin collagen; elastin, silk, fibrin, and fibrinogen; chitosan; bioresorbable polymers such as those made from polyglycolic acid; polylactic acid, polypropylenefumarate; polyethylene glycol; and mixtures thereof. In some embodiments, the bioresorbable material comprises demineralized bone matrix, such as is further described below regarding the carrier materials used in the compositions of this technology. In some embodiments the bioresorbable material comprises collagen.

The bioresorable material may be coated on a portion of the surface of the ceramic scaffold, or on substantially the entire surface of the ceramic scaffold. The bioresorbable material can fill, in whole or in part, pores on the surface of the ceramic scaffold. In this regard, the bioresorbable material can be applied to substantially all of the pores of the ceramic scaffold, or applied to pores in one or more selected regions of the ceramic scaffold.

The bone repair compositions may comprise from about 5% to about 60% of the ceramic scaffold by weight and from about 40% to about 95% of the bioresorbable material by weight. The ceramic scaffold can, for example, be present at a level of about 50% of the bone repair composition by weight, less than about 35% of the bone repair composition by weight, or less than about 25% of the bone repair composition by weight. The bioresorbable material can be present, for example, at less than about 90% of the bone repair composition by weight, or less than about 70% of the bone repair composition by weight. The bioresorbable material may be present at greater than 30% of the bone repair composition by weight, or greater than about 50% of the bone repair composition by weight. It is understood that factors such as the particular bone defect being repaired, the porosity of the scaffold, the particular bioresorbable material used, the composition of the carrier used, and the addition of any bioactive materials can provide variation among the above-disclosed ranges and that such variations are within the scope of the present teachings.

### Carrier

The compositions of the present technology comprise a carrier comprising denatured demineralized bone matrix. The carrier comprises from about 0.2% to about 40% of denatured demineralized bone, by weight of the carrier. In various embodiments, the carrier comprises from about 0.5% to about 25%, or from about 10% to about 20% of the denatured demineralized bone. The remainder of the carrier may comprise an aqueous solution such as water or saline.

The carrier is generally made by heating a solution of demineralized bone matrix and water. The demineralized bone may be made using methods among those known in the art. For example, bone may be collected from a donor source and can include the entire bone or bone fragments from cancellous or cortical bone. In some embodiments, the donor source is the subject to be treated with the composition of the present technology. In other embodiments, the donor source is not the subject being treated, but is from the same species (e.g., human). For example, the bone used to prepare the carrier for a human patient can be sourced from one or more human cadaveric donors. In various embodiments, the bone used to form the bone material and the carrier are from the same donor.

Once bone is obtained, adherent tissues can be removed by standard bone cleaning protocols. In various embodiments, the bone is milled into particles ranging from about 100 microns to about 2000 microns. Such milling includes any method of shaping the bone to a desired size by crushing, chopping, cutting, shaving, grinding, or pulverizing. In embodiments where several sizes of bone are be used, the milling process can be repeated and the respective bone portions can be reserved and assigned accordingly. Commercially available milling and sieving devices can be used or bone can be purchased in the form of an allograft matrix in the desired particle size or sizes.

Milled bone can be defatted by soaking or washing the bone in ethanol, to dissolve lipids. The ethanol bath also disinfects the bone by killing vegetative microorganisms and viruses. A further antiseptic step can include treatment of the milled bone with a hydrogen peroxide solution.

The milled bone is then demineralized using processes including those known in the art, such as by acidification or chelation. Acids used include inorganic acids such as hydrochloric acid, and organic acids such as peracetic acid. Chelating agents include disodium ethylenediaminetetraacetic acid (Na₂EDTA). The time required to demineralize the bone may vary depending on the concentration of acid or chelating agent used, the displacement or flow of the solution and the desired final concentration of calcium in the bone. For example, in an embodiment using hydrochloric acid at a concentration of 0.1 to 2.0 N, the bones can be soaked for up to 24 hours. The calcium or mineral concentration in the milled bone can be monitored by measuring the pH of the acid solution using a calcium specific electrode or a standard pH meter. In a preferred embodiment, the acid wash or soak ceases when the calcium concentration of the bone is less than 1%. After demineralization, the pH of the bone is adjusted by removing the acid with a deionized/distilled water or biocompatible buffer wash until the pH of the bone approximates that of the water.

To prepare the carrier, the demineralized bone is then added to an aqueous component such as water or a saline solution. The demineralized bone can be in a wet, moist or dry state or a combination of states. In some embodiments, from about 5 to about 25 grams, or from about 10 to about 20 grams, of demineralized bone is added per 100 grams of water or a saline solution. The specific amount is varied according to such factors as the desired composition, intended use, desired physical characteristics of the composition, and the size and shape of the bone used (e.g., chips, powder, fragments, etc.).

The water and demineralized bone material mixture is then heat-treated. Suitable heat treatments incorporate boiling, steaming, or the use of an oven. In various embodiments, autoclaving the adhesive results in the bone and water or saline mixture forming a gel or having a gel like consistency. Autoclaving is a thermal procedure, such as that used for sterilization, where the solution is placed in a sealed chamber and subjected to high temperature and pressure. Preferably, the water and demineralized bone mixture is autoclaved at a temperature of from about 100° C. to about 150° C., at a pressure of from about 10 psi to about 20 psi, for a period of a about 0 minutes to 2 hours. In a preferred embodiment, the mix is autoclaved at 121° C. under a pressure of 15 psi for 60 minutes. The duration of autoclaving can be adjusted depending upon the amount of demineralized bone and the amount and type of liquid used. The autoclaving demineralized bone is then cooled to about 5° C. for about 45 minutes to further gel the mixture and provide the proper viscosity. Methods among those useful herein are also disclosed in U.S. Pat. No. 6,576,249, Gendler et al., issued Jun. 10, 2003.

### Optional Materials

The compositions of the present technology optionally contain a bone material, such as bone powder, bone chips, bone shavings, and combinations thereof. Bone materials may comprise bone obtained from cortical, cancellous, and/or corticocancellous bone from a human or other animal subject. (See, e.g., U.S. Pat. No. 5,507,813, Dowd, et al., issued Apr. 16, 1996.) The bone may be allogenic, or autologous with the subject to be treated with the composition of the present technology. In various embodiments, about 100 grams of the carrier is mixed with from about 25 to about 45 grams, or from about 30 to 35 grams, of the bone material.

In one embodiment, the bone material is demineralized bone powder, which can be made following procedures as described above. In such embodiments, following pH adjustment as discussed above, the solution of demineralized bone is dried using suitable drying techniques. Drying techniques include lyophilization (freeze drying), vacuum drying, air drying, temperature flux drying, and molecular sieve drying. In some embodiments, the demineralized bone is lypophilized, by freezing the solution and evaporating the ice under a vacuum. The dried bone material preferably has a final moisture level of about less than 6%, as recommended by the American Association of Tissue Banks.

The demineralized bone powder preferably has a particle size of less than about 1500 microns. In various embodiments, the demineralized bone powder has a particle size less than about 1000 microns, less than about 850 microns, or less than about 710 microns. In one embodiment, the demineralized bone material has a particle size of less than about 100 microns.

The bone material can comprise bone chips. The bone chips can be natural or demineralized. The bone chips may range from about 750 to about 2000 microns in size. In one embodiment, the particles have a size of from about 750 to about 1500 microns.

The bone repair composition can also include other optional materials such as isolated tissue materials, bioactive agents, and combinations thereof. Isolated tissue materials comprise tissue material that has been extracted from a human or other animal subject and which, in some embodiments, has been subjected to processing. Examples of isolated tissue material include platelet-rich plasma, platelet-poor plasma and other blood components, bone marrow aspirate, concentrated bone marrow aspirate, and processed lipoaspirate cells. The isolated tissue material may contain hematopoietic stem cells, stromal stem cells, mesenchymal stem cells, endothelial progenitor cells, red blood cells, white blood cells, fibroblasts, reticulocytes, adipose cells, thrombocytes, and endothelial cells. The isolated tissue material may be autologous tissue, i.e. tissue from the subject to be treated with the composition of the present technology.

Optional bioactive agents include those that provide a therapeutic, nutritional, or cosmetic benefit. Such benefits may include repairing unhealthy or damaged tissue, minimizing infection at a treatment site, increasing integration of healthy tissue into the composition, and preventing disease or defects in healthy or damaged tissue. Bioactive agents include organic molecules such as proteins, peptides, peptidomimetics, nucleic acids, nucleoproteins, antisense molecules, polysaccharides, glycoproteins, lipoproteins, carbohydrates and polysaccharides, and synthetic and biologically engineered analogs thereof; living cells such as chondrocytes, bone marrow cells, and stem cells; viruses and virus particles; natural extracts; and combinations thereof. Examples of bioactive materials include antibiotics and other anti-infective agents, hematopoietics, thrombopoietics agents, antiviral agents, antitumoral agents (chemotherapeutic agents), antipyretics, analgesics, anti-inflammatory agents, therapeutic agents for osteoporosis, enzymes, vaccines, immunological agents and adjuvants, hormones, cytokines, growth factors, cellular attractants and attachment agents, gene regulators, vitamins, minerals and other nutritionals, and combinations thereof. The bioactive agent may include one or more cytokines, including isolated, synthetic, or recombinant molecules. Cytokines useful herein include growth factors such as transforming growth factor (TGF-beta), bone morphogenic proteins (BMP, BMP-2, BMP-4, BMP-6, and BMP-7), neurotrophins (NGF, BDNF, and NT3), fibroblast growth factor (FGF), granulocyte-colony stimulating factor (G-CSF), granulocyte-macrophage colony stimulating factor (GM-CSF), nerve growth factor (NGF), neurotrophins, platelet-derived growth factor (PDGF), erythropoietin (EPO), thrombopoietin (TPO), myostatin (GDF-8), growth differentiation factor-9 (GDF-9), basic fibroblast growth factor (bFGF or FGF-2), vular endothelial growth factor (VEGF), epidermal growth factor (EGF), insulin-like growth factors (IGF I, IFG-II), and combinations thereof.

### Methods of Manufacture:

The present technology also provides methods of making a bone repair composition comprising a porous ceramic scaffold having a bioresorbable coating and a carrier comprising denatured demineralized bone, Such methods include those comprising:

(a) mixing a demineralized bone material and water;

(b) heating the mixture of demineralized bone material and water to form a carrier;

(c) coating a bioresorbable material onto a surface of a porous ceramic scaffold to form a coated ceramic scaffold;

(d) forming a moldable composition comprising the carrier and the coated ceramic scaffold; and

(e) removing moisture from the moldable composition to provide the dried bone repair composition.

The mixing and heating steps of the method comprise methods as described above for making a carrier of the present technology. Accordingly, in some embodiments, the heating comprises autoclaving a mixture of demineralized bone material in saline or water.

The coating step may be conducted in any suitable manner so as to coat the bioresorbable material onto a surface of the ceramic scaffold, as discussed above. For example, demineralized bone matrix, collagen, or other bioresorbable material made liquid by dissolution or melting, may be coated onto the ceramic scaffold using techniques such as injection into the pores of the ceramic scaffold, submerging the ceramic scaffold into the liquid bioresorable material, spraying of the liquid bioresorbable material, and rolling or admixture of the ceramic scaffold into the liquid bioresorbable material. The bioresorbable material may also be vacuum-coated onto the ceramic scaffold. In such embodiments, the ceramic scaffold is placed under a vacuum and a liquid of the bioresorbable material, such as the demineralized bone gel is drawn into the pores of the scaffold via the vacuum pressure.

Depending on the desired thickness of the bioresorbable material coating layer, a single technique or a combination of techniques can be employed. For example, when applying an ultra thin layer (from about 10 nm to about 5 mm) of bioresorbable coating a spray type application utilizing a fine mist can provide greater control. When a thicker layer is preferred, rolling or submerging the ceramic scaffold in the bioresorbable material can provide a thick layer more quickly than other techniques.

The bioresorable material can then be dried onto the ceramic scaffold. The drying is performed at a temperature and for a duration sufficient to provide secure attachment of the bioresorbable material to the surface of the ceramic scaffold.

A moldable composition is then formed by admixing the coated ceramic scaffold with the carrier to form a paste or moldable material. This may be done using any suitable technique. For example, this mixing can be performed when the carrier is essentially in a liquid state or when it has formed a gelatinous mass after cooling. The mixing can be performed in a mold for making a formed composition, as discussed below.

The moldable composition is then dried so as to remove moisture. Drying may be accomplished by any suitable method, including those discussed above regarding the production of optional bone materials. In one embodiment, the moldable composition is lyophilized. Preferably, the moisture level upon drying is less than 6% total moisture by total weight of the moldable composition.

Optionally, the moldable composition is formed into a formed composition prior to drying. Formed compositions have non-random shapes, preferably of a size and dimension suitable for implantation to the site of a bone defect. In various embodiments, the shapes can be specifically formed for a desired end-use application, as a site-specific preform.

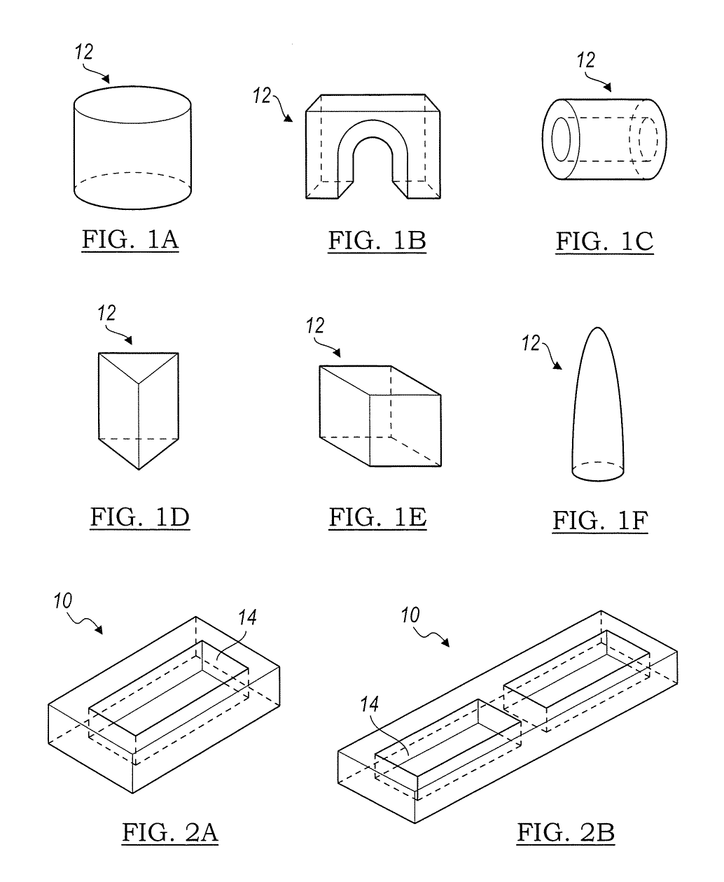

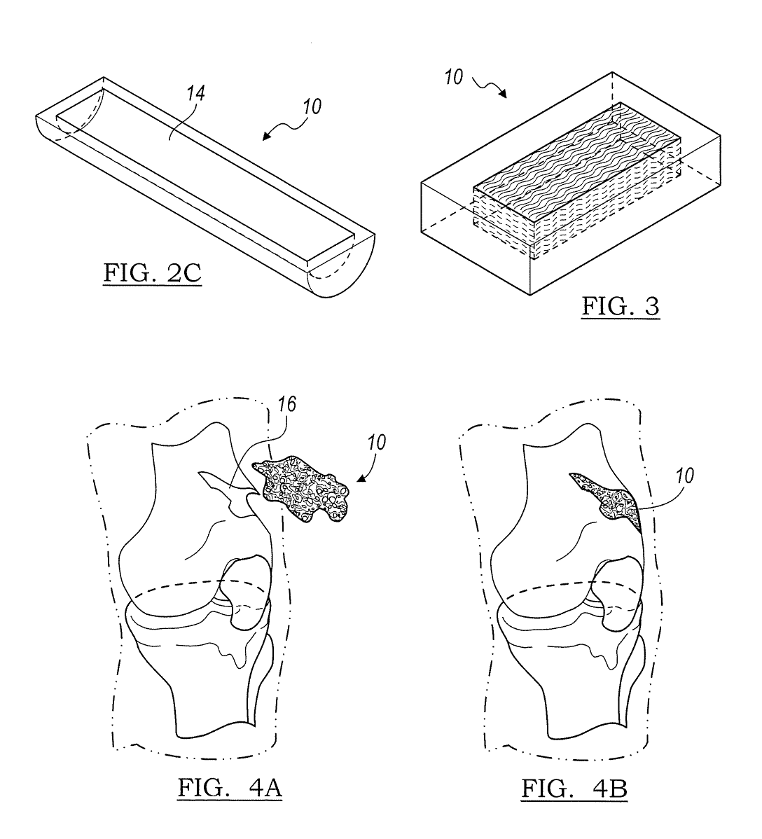

Examples of formed compositions 10 are depicted in FIGS. 1 through 4B. Formed compositions include cylinders, troughs, sleeves, triangular prisms, rectangular prisms, and ellipsoidal forms as depicted in FIGS. 1A through 1F, respectively, as well as sheets, rods, and shapes having other cross-sectional shapes. As shown in FIGS. 2A through 2C, the ceramic scaffold 12 can be in the shape of a trough or tray, including at least one recessed region or channel 14 for filling with a bone material or bioactive material. The ceramic scaffold 12 can also be a free-form or irregular, such as depicted in FIGS. 4A and 4B.

Bone materials, bioactive materials or other optional materials, such as those described above, can also be added during the process for making the bone repair composition. In various embodiments, the optional materials are added during or after the step of forming the moldable composition. Optional materials may also be added during the step of making the carrier, and the step of coating the bioresorbable coating on to a surface of the ceramic scaffold. The timing of addition may be important in some embodiments, however, because the properties of the material can be compromised. For example, calcium-containing bone materials are preferably not added prior or during the manufacture of the demineralized bone matrix.

### Methods of Repairing Bone Defects

The present technology also provides methods for repairing a bone defect. Bone defects include any area of bone tissue that is inadequate for cosmetic or physiological purposes. Bone defects may be caused by birth defect, trauma, disease, decay, or surgery. For example, bone repair compositions of the present technology can be used to correct bone defects resulting from orthopedic, neurosurgical, plastic or reconstructive surgery, periodontal, and endodontic procedures. Specific examples include repair of simple and compound fractures and non-unions, external and internal fixations, joint reconstructions such as arthrodesis, general arthroplasty, cup arthroplasty of the hip, femoral and humeral head replacement, femoral head surface replacement and total joint replacement, repairs of the vertebral column including spinal fusion and internal fixation, tumor surgery, e.g. deficit filling, discectomy, laminectomy, excision of spinal cord tumors, anterial cervical and thoracic operations, repair of spinal injuries, scoliosis, lordosis and kyphosis treatments, intermaxillary fixation of fractures, mentoplasty, temporomandibular joint replacement, alveolar ridge augmentation and reconstruction, inlay bone grafts, implant placement and revision, and sinus lifts.

Methods of the present technology include those for repairing a bone defect comprising implanting at the site of the defect a bone repair composition comprising a porous ceramic scaffold having a bioresorbable coating, and a carrier comprising denatured demineralized bone: In some embodiments, methods comprise (a) providing a dehydrated bone repair composition comprising a porous ceramic scaffold having a bioresorbable coating, and a carrier comprising denatured demineralized bone; b) hydrating the bone repair composition; and (c) implanting the bone repair composition the site of the defect.

In embodiments where the bone repair composition is smaller than the defect, the surgeon may place a single bone repair composition or several bone repair compositions in the defect site and manipulate them appropriately by hand or with a surgical tool. The bone repair composition can be designed to fill and partially wrap around a defect site. The surgeon can match the contour of the bone repair composition with the contour of the defect and insert the bone repair composition into the void. In an embodiment where several bone repair compositions are used to provide strength of a defect, the bone repair compositions can be oriented to maximize strength of the repair.

As depicted in FIGS. 4A and 4B, the formed bone repair composition 10 can be used to augment the defect 16 in the femur above the patella. Because this region of the femur is subject to high load and stress, the formed bone repair composition 10 is placed into the defect 16 to provide supplemental strength. Regenerated tissue may grow into the formed bone repair composition 10 and replace the demineralized bone matrix and any other bioactive or resorbable materials in the pores. Although the bone defect is depicted on a knee related defect, the methods and materials may be used for any defect.

The formed bone repair composition 10 can also be shaped for specific uses. As shown in FIGS. 1A through 3, exemplary bone repair compositions 10 are trough-style and contain a channel 14 or multiple channels. The channels 14 can be used to contain a bone-building material. For example, the channel(s) 14 can be filed with autograft bone chips, bone graft substitute, or any other bone-building material disclosed herein. The channel 14 may also be useful for facilitating in-growth of new bone.

Referring to FIG. 2, specific uses of the trough-style augments 10 include posterolateral fusions or high tibial osteotomy for example. The rounded trough-style formed bone repair compositions 10 depicted in FIG. 2C can be advantageously used in spinal applications.

A site-specific bone repair composition 10 can have the dimensions of the defect 16 to be filled and does not require additional manipulation in the operating room. The dimensions can be acquired using an x-ray of the site of the defect as a reference for size and shape. The x-ray can be scaled to the appropriate dimensions for the cast. Depending on the quantity and type of bone defect repairs required, a plurality of generic and site-specific bone repair composition 10 can be used during the surgery. Additionally, site-specific bone repair compositions 10 can conform to the geometry of the adjacent host bone to facilitate efficient incorporation of new bone.

In an embodiment where the bone repair composition 10 is substantially or completely dried, the bone repair composition 10 can be reconstituted or hydrated. The solution used to hydrate the bone repair composition 10 can include, but is not limited to, water, saline, whole blood, blood fractions, bone marrow aspirates, derivatives thereof, and mixtures thereof. In one embodiment, adding water to the dried bone can be achieved by adding blood to the bone repair composition 10. Blood fractions include blood components such as red blood cells, white blood cells, plasma, plasma fractions, plasma serum, platelet rich plasma, platelet poor plasma, blood proteins, thrombin, coagulation factors, and mixtures thereof.

The bone repair composition 10 can be hydrated after implanting at the defect 16. Ambient fluids such as blood may be absorbed after a few minutes. Extra corpus fluids, including but not limited to, saline, water or a balanced salt solution (140 mm NaCl, 5.4 mm KCl, pH 7.6) can be used to expedite the hydration. Preferably, the hydrated bone repair composition 10 maintains a cohesive, initial shape for at least 30 minutes after hydration without crumbling or becoming misshapen.

The examples and other embodiments described herein are exemplary and not intended to be limiting in describing the full scope of compositions and methods of this invention. Equivalent changes, modifications and variations of specific embodiments, materials, compositions and methods may be made within the scope of the present invention, with substantially similar results.

What is claimed is:

1. A formed composition for application to a bone surface of a human or animal subject, comprising:
(a) a porous ceramic scaffold having a bioresorbable coating; and
(b) a carrier comprising denatured demineralized bone.

2. The bone repair composition of claim 1, wherein the porous ceramic scaffold comprises a material selected from the group consisting of hydroxyapatite, calcium phosphates, calcium carbonates, calcium sulfates, and combinations thereof.

3. The bone repair composition of claim 2, wherein the porous ceramic scaffold comprises tricalcium phosphate.

4. The bone repair composition of claim 2, wherein the porous ceramic scaffold comprises coralline hydroxyapatite.

5. The bone repair composition of claim 1, wherein the ceramic scaffold has a pore size of from about 300 to about 800 microns

6. The bone repair composition of claim 5, wherein the ceramic scaffold has a median pore size of about 500 microns.

7. The bone repair composition of claim 1, additionally comprising a bone material selected from the group consisting of bone powder, bone chips, bone shavings, and combinations thereof.

8. The bone repair composition of claim 1, wherein the bone material comprises a demineralized bone powder.

9. The bone repair composition of claim 8, wherein the demineralized bone powder has a particle size of less than about 850 micrometers.

10. The bone repair composition of claim 1, wherein the bioresorable coating is selected from the group consisting of demineralized bone matrix, gelatin, collagen, and mixtures thereof.

11. The bone repair composition of claim 10, wherein the bioresorbable coating is selected from the group consisting of demineralized bone matrix, collagen, and mixtures thereof.

12. The bone repair composition of claim 11, wherein the bioresorbable coating comprises collagen.

13. The bone repair composition of claim 1, wherein the composition is formed into a shape suitable for administration to a bone defect.

14. The bone repair composition of claim 13, wherein the shape is selected from the group consisting of sheets, patches, blocks, rings, discs, cylinders, troughs, or site-specific pre-forms.

15. A method for making a bone repair composition comprising:
(a) mixing a demineralized bone material and water;
(b) heating the mixture of demineralized bone material and water to form a carrier;
(c) coating a bioresorbable material onto a surface of a porous ceramic scaffold to form a coated ceramic scaffold;
(d) forming a moldable composition comprising the carrier and the coated ceramic scaffold; and
(e) removing moisture from the moldable composition to provide the dried bone repair composition.

16. The method of claim 15, wherein the porous ceramic scaffold comprises a material selected from the group consisting of hydroxyapatite, tricalcium phosphate, calcium phosphates, and combinations thereof.

17. The method of claim 15, wherein heating the mixture comprises autoclaving.

18. The method of claim 17, wherein the autoclaving is conducted at a temperature of from about 100° C. to about 150° C., at a pressure of from about 10 psi to about 20 psi for from about 0 minutes to about 2 hours.

19. The method of claim 15, wherein removing the moisture utilizes a drying technique selected from lyophilizing, vacuum drying, air drying, temperature flux drying, and molecular sieve drying.

20. The method of claim 15, wherein the moldable composition further comprises a bone material selected from the group consisting of bone powder, bone chips, and combinations thereof.

21. The method of claim 15, wherein the moldable composition is formed into a shape selected from the group consisting of sheets, patches, blocks, rings, discs, cylinders, troughs, or site-specific pre-forms.

22. A formed bone repair composition comprising:
(a) a ceramic scaffold, having porosity of from about 150 microns to about 800 microns, comprising hydroxyapatite, tricalcium phosphate, calcium phosphates, calcium carbonates, calcium sulfates, and combinations thereof;
(b) a bone material selected from the group consisting of: bone powder, bone chips, bone shavings, and combinations thereof, and
(c) a carrier comprising denatured demineralized bone;
wherein a surface of the ceramic scaffold is coated with a bioresorable material selected from the group consisting of demineralized bone, gelatin, collagen, and mixtures thereof;
and the composition is formed into a shape suitable for administration to the bone.

23. The formed bone repair composition according to claim 22, wherein the ceramic scaffold comprises coralline hydroxyapatite.

24. The formed bone repair composition according to claim 22, wherein the bone material comprises demineralized bone powder.

25. The bone repair composition of claim 22, wherein the shape is selected from the group consisting of sheets, patches, blocks, rings, discs, cylinders, troughs, or site-specific pre-forms.
